(19) United States
(12) Patent Application Publication
(10) Pub. No.: US 2024/0016419 A1
(43) Pub. Date: Jan. 18, 2024

(54) ELECTROCHEMICAL GLUCOSE SENSING BY EQUILIBRIUM GLUCOSE BINDING TO GENETICALLY ENGINEERED GLUCOSE BINDING PROTEINS

(71) Applicant: Cercacor Laboratories, Inc., Irvine, CA (US)

(72) Inventors: Anando Devadoss, Irvine, CA (US); Venkatramanan Krishnamani, Irvine, CA (US)

(21) Appl. No.: 18/352,850

(22) Filed: Jul. 14, 2023

Related U.S. Application Data
(60) Provisional application No. 63/368,760, filed on Jul. 18, 2022.

(51) Int. Cl. A61B 5/145; A61B 5/1486

(52) U.S. Cl. CPC A61B 5/14532; A61B 5/1486

(57) ABSTRACT

Aspects of the present disclosure provide devices and methods capable of optimizing in vivo electrochemical measurement of a molecule of interest, for example glucose. Such aspects may include an engineered binding protein, for example an engineered glucose binding protein. The engineered binding protein may change conformation in response to binding or unbinding to a ligand and/or analyte, for example glucose. Such conformational changes may either expose or occlude a redox molecule attached to the binding protein. When exposed, the redox molecule may generate a redox signal dependent upon the binding protein's conformational state. The redox signal may be measurable, for example by cyclic voltammetry. The protein may be incorporated into a sensor that can be used as an implantable continuous glucose monitoring device.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. For example, this application claims priority to U.S. Provisional Application No. 63/368,760, filed Jul. 18, 2022, the entire content of which is incorporated by reference herein in its entirety for all purposes and forms a part of this specification.

### BACKGROUND

### Field

The present disclosure relates to continuous glucose monitoring (CGM). More specifically, it relates to CGM sensors.

### Description of the Related Art

Monitoring of blood glucose concentration levels has long been critical to the management and care of diabetes mellitus. Current blood glucose monitors involve a chemical reaction between blood or serum and a test strip, requiring an invasive extraction of blood via a lancet or pinprick. Small handheld monitors have been developed to enable a patient to perform this procedure anywhere, at any time. But the inconvenience of this procedure—specifically the blood extraction, the pain associated with the procedure and the use and disposition of lancets and test strips—has led to a low level of compliance. As such, it is desirable to continuously monitor the concentration of glucose level in the human body.

A first generation of electrochemical continuous glucose monitoring (CGM) sensor was developed based on Clark-type amperometric detection. Such CGM sensor measures the current generated by the electrochemical oxidation of hydrogen peroxide (H₂O₂) at the surface of either an activated platinum or a platinum/iridium (90:10 Pt:Ir) electrode. Hydrogen peroxide (H₂O₂) is a byproduct of oxidation (loss of electrons) of glucose by the enzyme glucose oxidase (GOx). Each molecule of glucose oxidized by GOx generates one molecule of H₂O₂ as a byproduct, which acts as a proxy for measuring the glucose concentration. The H₂O₂ fraction that diffuses inward (towards the electrode) gets electrochemically oxidized into oxygen at the Pt surface thereby generating current to measure glucose concentration.

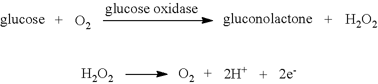

CGM sensors based on electrochemical methods may be capable of detecting glucose by indirect measurement of a molecule that is generated during the enzymatic reaction. The enzymatic product may be, as examples, hydrogen peroxide, an artificial mediator, and/or the reduced enzyme co-factor itself. The common aspect for all these measurements is the application of an electrochemical potential sufficient to (re)oxidize these molecules to generate a current.

### SUMMARY

For purposes of summarizing the disclosure, certain aspects, advantages and novel features of several devices, systems, and methods have been described herein. It is to be understood that not necessarily all examples of the present disclosure are disclosed herein. Thus, the devices, systems, and methods disclosed herein can be embodied or carried out in a manner that achieves or optimizes one advantage or group of advantages as taught herein without necessarily achieving other advantages as can be taught or suggested herein.

Aspects of the present disclosure relate to a sensor. The sensor may include a first binding protein configured to have a first conformation and a second conformation, where electrons are transferred to the electrode surface when the first binding protein is in the first conformation, the first binding protein comprising a binding site, and whether the first binding protein is in the first conformation or the second conformation depends, at least in part, on whether there is an analyte bound to the binding site; and a sensing electrode. In some aspects, the sensor may include a redox mediator configured to create the electrical signal, where the redox mediator is not active or partially active when the first binding protein is in the first conformation, the redox mediator is active or relatively more active when the first binding protein is in the second conformation. In some aspects, the sensor of the present disclosure may include a redox mediator configured to create the electrical signal, and an affecter molecule configured to shift the activity of the redox mediator when the redox mediator and the affecter molecule are in proximity; wherein the redox mediator and affecter molecule are not in proximity when the first binding protein is in the first conformation, the redox mediator and affecter molecule are in proximity when the first binding protein is in the second conformation. In some aspects, the first binding protein may include a glucose binding protein. In some aspects, the glucose binding protein may have a binding constant between 100 nM to 200 mM. In some aspects, the glucose binding protein may have a binding constant between 2 mM to 22 mM. In some aspects, the analyte includes glucose. In some aspects, the binding site includes a glucose binding site. In some aspects, the sensor includes a second binding protein, wherein the first binding protein and second binding protein do not have the same binding constant. In some aspects, the redox mediator may be a Ru(II) cofactor, a porphyrin, or a quinone. In some aspects, the sensing electrode includes a nanowire network. In some aspects, the sensing electrode includes a hydrogel. In some aspects, the sensing electrode includes a polymer matrix.

Aspects of the present disclosure relate to a method of measuring the concentration of an analyte. The method may include exposing a binding protein to a sample fluid, the binding protein configured to have a first conformation and a second conformation, wherein whether the binding protein is in the first conformation or second conformation is dependent, at least in part, on whether there is an analyte bound to a binding site of the binding protein; and measuring an electrical signal dependent, at least in part, on whether the binding protein is in the first conformation or second conformation. In some aspects, measuring the electrical signal includes using an electrode comprising a nanowire network. In some aspects, the analyte comprises glucose. In some aspects, the binding protein includes a glucose binding protein. In some aspects, the binding site includes a glucose binding site. In some aspects, the electrical signal may be generated by a redox mediator. In some aspects, the redox mediator is not active when the binding protein is in the first conformation. In some aspects, the redox mediator is partially active when the binding protein is in the first conformation. In some aspects, the redox mediator is active when the binding protein is in the second conformation. In some aspects, the mediator has higher activity in the second conformation than the first conformation. In some aspects, the electrical activity of the redox mediator is dependent, at least in part on proximity to an affecter molecule, where the redox mediator and affecter molecule are not in proximity when the binding protein is in the first conformation, and the redox mediator and affecter molecule are in proximity when the binding protein is in the second conformation.

### BRIEF DESCRIPTION OF THE DRAWINGS

The above-mentioned aspects, as well as other features, aspects, and advantages of embodiments of the present disclosure will now be described in connection with various implementations, with reference to the accompanying drawings. The illustrated implementations are merely examples and are not intended to be limiting. Throughout the drawings, similar symbols typically identify similar components, unless context dictates otherwise.

FIG. 1A schematically illustrates the electrons transfer of a typical enzymatic glucose sensor involving hydrogen peroxide.

FIG. 1B schematically illustrates the electrons transfer of a typical enzymatic glucose sensor involving a mediator.

FIG. 2 illustrates a ribbon diagram of open and closed conformations of glucose binding protein.

FIG. 3A illustrates a cartoon diagram showing the various parts of a glucose binding protein.

FIG. 3B illustrates a cartoon diagram showing the various parts of a glucose binding protein when glucose is bound.

FIG. 4A illustrates a cartoon diagram showing the various parts of an engineered glucose binding protein.

FIG. 4B illustrates a cartoon diagram showing the various parts of an engineered glucose binding protein when glucose is bound.

FIG. 5 schematically illustrates usage of an engineered glucose binding protein with an electrode to generate a detectable signal.

FIGS. 6A-6C illustrate an example aspect of engineered glucose binding proteins embedded within a conductive hydrogel at varying levels of glucose concentration.

### DETAILED DESCRIPTION

Aspects of the disclosure will now be set forth in detail with respect to the figures and various examples. One of skill in the art will appreciate, however, that other configurations of the devices and methods disclosed herein will still fall within the scope of this disclosure even if not described in the same detail. Aspects of various configurations discussed do not limit the scope of the disclosure herein, which is instead defined by the claims following this description.

Aspects of the present disclosure provide devices and methods capable of optimizing in vivo electrochemical measurement of a molecule of interest, for example glucose. Such aspects may include an engineered binding protein, for example an engineered glucose binding protein. The engineered binding protein may change conformation in response to binding or unbinding to a ligand and/or analyte, for example glucose. Such conformational changes may either expose or occlude a redox molecule attached to the binding protein. When exposed, the redox molecule may generate a redox signal dependent upon the binding protein's conformational state. The redox signal may be measurable, for example by cyclic voltammetry, chronoamperometry, and/or electrochemical impedance spectroscopy.

### Definition

As used herein, common abbreviations are defined as follows:
° C. Temperature in degrees Centigrade
CE Counter electrode
CGM Continuous glucose monitoring
DAN Diamino naphthalene
GOx Glucose oxidase
H₂O₂ Hydrogen peroxide
oPD ortho-Phenylenediamine
mPD meta-Phenylenediamine
PBS Phosphate buffered saline
pPD para-Phenylenediamine
PmPD Poly(meta-phenylenediamine)
PoPD Poly(ortho-phenylenediamine)
PpPD Poly(para-phenylenediamine)
PVA Polyvinyl alcohol
RE Reference electrode
WE Working electrode

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. The terms “comprising,” “including,” “having,” and the like are synonymous and are used inclusively, in an open-ended fashion, and do not exclude additional elements, features, acts, operations, and so forth. That is, the above terms are to be interpreted synonymously with the phrases “having at least” or “including at least.” For example, when used in the context of a process, the term “comprising” means that the process includes at least the recited steps, but may include additional steps. When used in the context of a device, the term “comprising” means that the device includes at least the recited features or components, but may also include additional features or components. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The term “and/or” as used herein has its broadest least limiting meaning which is the disclosure includes A alone, B alone, both A and B together, or A or B alternatively, but does not require both A and B or require one of A or one of B. As used herein, the phrase “at least one of” A, B, “and” C should be construed to mean a logical A or B or C, using a non-exclusive logical “or”.

The term “temperature independent” as used herein, means that the reading or measurement of the glucose level by the glucose monitoring device or the response of the glucose sensor is not affect or not substantially affected by the change of temperature. In other words, the sensor is insensitive the change of temperature (e.g., change of body temperature as a result of physiological conditions such as hypothermia and hyperpyrexia). In some embodiments, the temperature independent property of the glucose monitoring device is maintained within the operating temperature range of the device (e.g., from about 30° C. to about 45° C., from about 33° C. to about 43° C., from about 35° C. to about 41° C., or from about 36° C. to about 40° C. In some embodiments, the change of temperature (per ° C.) results in less than 5%, 4%, 3%, 2%, 1%, 0.5%, 0.1% or 0.01% change in the response of the sensor, or the measurement/reading provided by the device, when all the other parameters remain the same (e.g., the glucose concentration is constant).

Any methods disclosed herein need not be performed in the order recited. The methods disclosed herein include certain actions taken by a practitioner; however, they can also include any third-party instruction of those actions, either expressly or by implication.

### Enzymatic Glucose Sensors

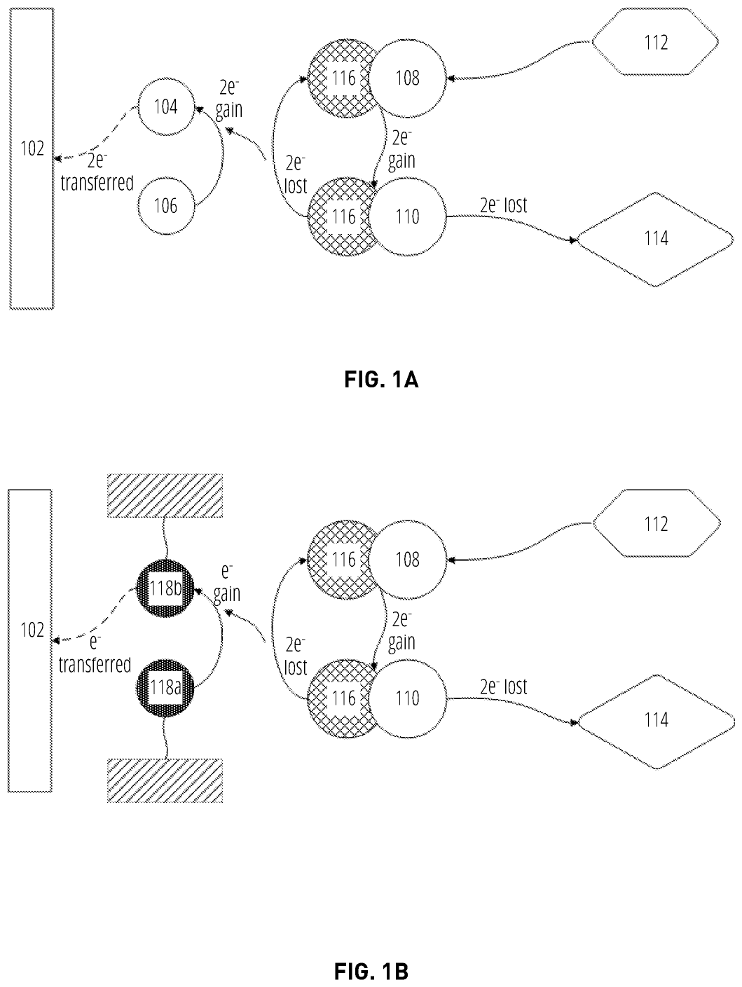

Certain subcutaneously implanted glucose sensors rely on enzymatic reaction that specifically converts glucose molecules and, in the process, produces a byproduct that is electroactive, for example hydrogen peroxide or artificial mediators. The byproduct may then be oxidized at the working electrode to generate a current that may be measured. As illustrated by FIGS. 1A and 1B, the electrons transferred from enzymatic oxidation of glucose is “captured” by the electrochemical sensor. FIG. 1A depicts one example implementation in which electrons are transferred from glucose 112 to the electrode 102 via hydrogen peroxide 104. A glucose 112 interacts with enzyme 116 and FAD 108, transferring electrons off the glucose 112. This acquisition of electrons converts FAD 108 to FADH₂ 110. Glucose 112 is converted to gluconic acid 114 by the enzyme 116. The enzyme 116 transfers two electrons from FADH₂ 110 to oxygen 106 (02), creating hydrogen peroxide 104. The FADH₂ 110 is converted back to FAD 108. The electrode 102 may apply a potential to oxidize hydrogen peroxide 104, transferring electrons from the hydrogen peroxide 104. This transfer of electrons from the hydrogen peroxide 104 to the electrode 102 may be detected.

FIG. 1B illustrates a second implementation in which transfer of electrons to the electrode 102 is accomplished via an artificial mediator 118. Much of the cycle is the same, but the enzyme 116 transfers one or more electrons to an artificial mediator 118, converting mediator 118a to mediator having negative charge 118b, rather than transferring electrons to oxygen 106 to create hydrogen peroxide 104. The electrode 102 may apply a potential to the mediator having negative charge 118b, oxidizing the mediator having negative charge 118b and transferring one or more electrons to the electrode. This transfer of electrons from the mediator having negative charge 118b to the electrode 102 may be detected.

However, both enzyme-based sensor designs depicted in FIGS. 1A and 1B may have shortcomings. For example, both sensors measure a constantly changing local glucose concentration. The local glucose concentration may change due to, for example, the enzymatic action of the sensor. The consumption of the analyte being measured by the enzyme may result in particularly inaccurate estimates of glucose concentration when measuring low glucose concentrations. Further, enzyme activity gradually decreases over time. For the implementations of FIGS. 1A-1B, this results in limited sensor life. The enzymatic conversion of glucose is also dependent on other molecular species. The conversion of glucose only occurs in the presence of a cofactor, FAD and substrates, oxygen and glucose. A low concentration of cofactor or glucose may thus reduce the conversion of glucose. Lastly, one of the byproducts of enzyme conversion of glucose is hydrogen peroxide. Hydrogen peroxide can also be produced by physiological phenomena, such as inflammation or other processes. Hydrogen peroxide is a highly reactive molecule which may degrade the enzyme and/or other chemical and biochemical sensor components. Thus, the presence of hydrogen peroxide may further reduce sensor life.

There are some drawbacks that are specific to implementations in accordance with FIG. 1A. For example, in the subcutaneous tissue, the number of glucose molecules far exceeds the amount oxygen molecules. For example, there may be up to about a 1000-fold difference between the number of glucose molecules and the number of oxygen molecules. Contrastingly, the enzymatic conversion reaction requires equimolar amounts of both glucose and oxygen. Sensors may thus be designed to proportionally reduce the glucose concentration within the sensor, where the enzyme is located. This may result in low signal, since relatively few glucose molecules are available to react via the enzymatic conversion. For example, signal may range between 1 pA to 10 nA. Such low currents may necessitate highly sensitive electronic circuits, which may be expensive.

Further, the signal, in the form of an electric current, is generated by oxidizing hydrogen peroxide at an electrode. The electrode may need to be held at a relatively high potential, for example +0.6 V vs Ag/AgCl to generate this signal in the presence of hydrogen peroxide. At this working potential, there are a multitude of endogenous and exogenous molecules that may also be oxidized at the electrode. These molecules are referred to herein as electrochemical interferents. Oxidation of electrochemical interferents may generate additional electrical current which may combine with and/or convolute the signal due to glucose. To eliminate these electrochemical interferents, many strategies have been developed, including but not limited to size-based filtering and/or charge-based filtering. However, these strategies typically require one or more additional layers shielding the working electrode. Additional layers may increase the complexity of manufacturing and may require extensive biocompatibility studies to demonstrate safety of the device.

Likewise, there are some drawbacks that are specific to implementations in accordance with FIG. 1B. For example, it may be difficult to identify a suitable redox mediator. It may be desirable that such a mediator has relatively low operational potential so that the currents from electrochemical interferents are minimal. Additionally, the redox mediator should not disintegrate with constant operation under the applied potential. Disintegration of the redox mediator may lead to errors in estimation of glucose concentration due to loss in current. Additionally, disintegration of the redox mediator may cause safety issues, as the integrated molecules can leach into the surrounding biofluid.

Herein, a novel configuration of electrochemical analyte sensor is disclosed. The sensor in accordance with the present disclosure does not require the enzymatic conversion of the analyte. Rather, the equilibrium binding of the analyte to a binding protein is exploited to generate a measurable signal.

### Binding Proteins

Suitable binding proteins may bind with specificity to a target analyte. Additionally, suitable binding proteins may undergo a conformational change in response to binding to the analyte.

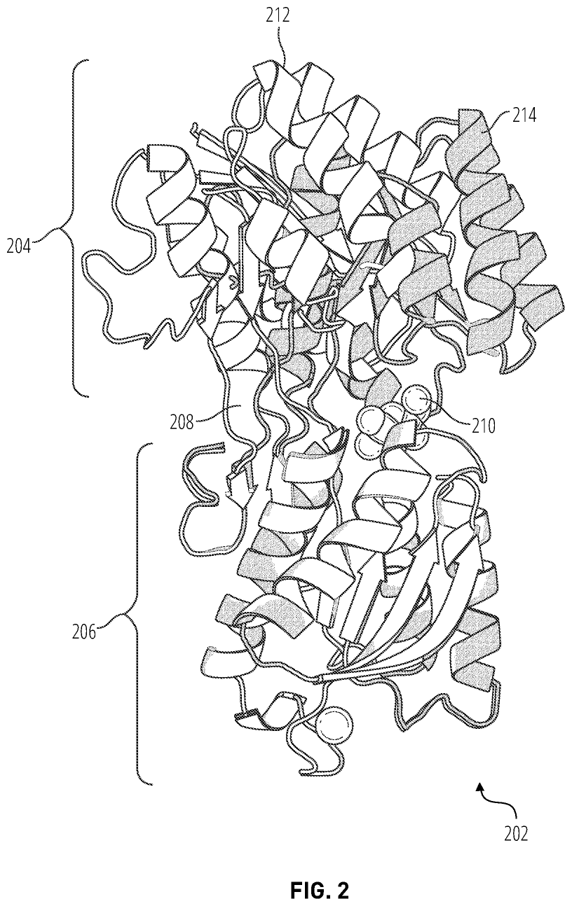

One example binding protein is glucose binding protein. Glucose binding protein (GGBP), from E. coli (Uniprot ID: P0AEE5), is a member of protein family called periplasmic binding proteins (Pfam ID: PF18610), mainly found in bacteria. Members of this family of proteins have a specific affinity to a ligand and/or analyte. As an example, GGBP has a specific affinity to glucose and/or galactose. The structure of GGBP 202 is illustrated as a ribbon diagram in FIG. 2. Generally, GGBP includes two large protein domains, the N-terminal domain 204 and the C-terminal domain 206, that are linked to each other by a flexible hinge 208. Other example binding proteins include Vitamin-D binding protein, Odorant-binding protein (from olfactory receptors), Calcium binding protein, or any-hormone binding proteins/receptors. Additionally or alternatively, portions of such binding proteins may be suitable for inclusion in an electrochemical analyte sensor of the present disclosure. For instance, a portion of the binding protein that changes conformation substantially in response to binding/unbinding an analyte may be suitable for inclusion. Suitable analytes may include sugars such as glucose, galactose, lactose, specific nucleotide sequences, insulin, growth hormones, retinol, calcium, iron, or other molecules present in physiology. Further, suitable binding proteins may be engineered, for instance by directed-evolution, rational design, or de novo design techniques. As an example, a naturally-occurring binding protein or binding protein fragment, for instance any of the examples mentioned above, may be chosen as a starting point for modification to create a binding protein (referred to herein as an engineered binding protein) for inclusion in a sensor of the present disclosure. Modification of a binding protein is discussed herein with reference to GGBP. A binding protein may be modified to, for example, alter analyte binding kinetics, alter a conformational change, alter the type of analyte to which the protein is capable of binding, or alter the size and/or shape of the binding protein (which in turn may thereby change potential steric effects and/or diffusion rate of the protein).

Certain aspects of GGBP may make it useful for inclusion in a sensor in accordance with present disclosure. Firstly, GGBP binds to glucose and/or galactose molecules with very high affinity and specificity. Secondly, GGBP binding to glucose is reversible and does not chemically convert the analyte. Thirdly, when glucose binds GGBP, there is a significant conformation change in the protein. This conformation change is illustrated in FIG. 2. The unbound conformation 212 is illustrated without shading. The bound conformation 214 is illustrated with shading. Note that, in bound conformation 214, the N-terminal domain 204 and C-terminal domain 206 close around the glucose 210.

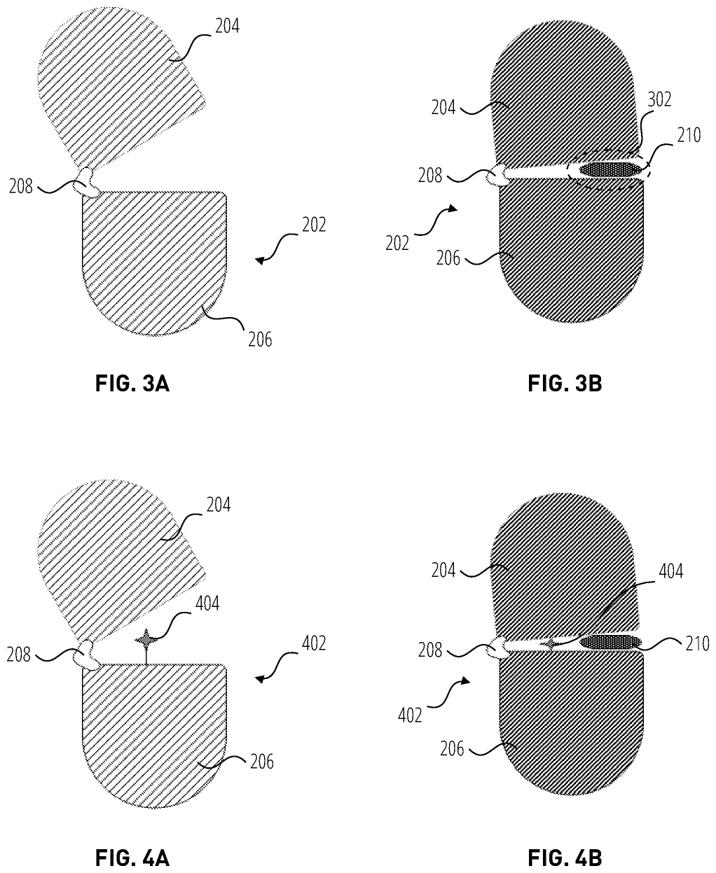

The conformational change is also illustrated schematically in FIGS. 3A and 3B. FIG. 3A illustrates the unbound conformation of GGBP 202 while FIG. 3B illustrates the glucose-bound conformation of GGBP 202. Glucose binds to the glucose binding site 302. When bound to glucose, the N-terminal domain 204 and the C-terminal domain 206 come closer to each other and the motion at the hinge 208 is limited in comparison to the unbound form. In the glucose-bound form of GGBP (FIG. 3B), portions of the protein in proximity to the hinge area may be more exposed or less exposed than they are in the glucose-unbound form (FIG. 3A). Structurally, the bound conformation is relatively rigid while the unbound conformation is more flexible about the hinge. The conformational changes of GGBP are described in greater detail by Borrok, M. J., et al., Conformational changes of glucose/galactose-binding protein illuminated by open, unliganded, and ultra-high-resolution ligand-bound structures, Protein Sci. 16, 1032-41 (2007), incorporated herein by reference.

As disclosed herein, the conformational change in GGBP when bound to glucose may be exploited to create a concentration-dependent biosensor that can propagate an electrochemical signal.

Wild-type GGBP binds to glucose at very high affinity, with a binding constant of 0.2 μM. Realistic physiological glucose concentrations range between approximately 2 mM to 22 mM. Within this range, close to 100% of wild-type GGBP would be bound to glucose. As disclosed herein, GGBP may be modified to shift and/or alter binding affinity to glucose using methods described in the art. For example, changing Alanine (A) residue at the 213 position in GGBP to an Arginine (R) residue (A213R) results in a binding constant shift of 5000-fold, from 0.2 μM to 1 mM. This mutation, among others, is discussed by Amiss, T. J., et al., Engineering and rapid selection of a low-affinity glucose/galactose-binding protein for a glucose biosensor, Protein Sci. 16, 2350-59 (2007), incorporated herein by reference.

GGBP has 332 amino acids in its protein sequence, of which 16 amino acids are situated in the glucose binding site 302. Two related strategies may allow fine-tuning of GGBP's glucose binding affinity to better match the physiological glucose range.

The first strategy involves genetically engineering some, or all, of the 16 amino acid positions of the glucose binding site 302. For example, any of those 16 amino acid positions may be changed to a different amino acid of the 20 standard amino acid choices. Alterations to these 16 binding pocket amino acids may alter a GGBP's glucose binding affinity such that its binding constant is within, or close to, the relevant physiological range.

The second strategy involves genetically engineering several different modified GGBPs. Multiple mutated GGBPs having different binding constants can be mixed in a sensor to achieve sensitivity across the range of glucose concentration found in physiology. As an illustrative example, a set of GGBPs with binding constants of 2 mM, 4 mM, 8 mM and 16 mM may provide coverage over physiologically relevant glucose concentrations. Other sets of GGBPs having other binding constants may be suitable.

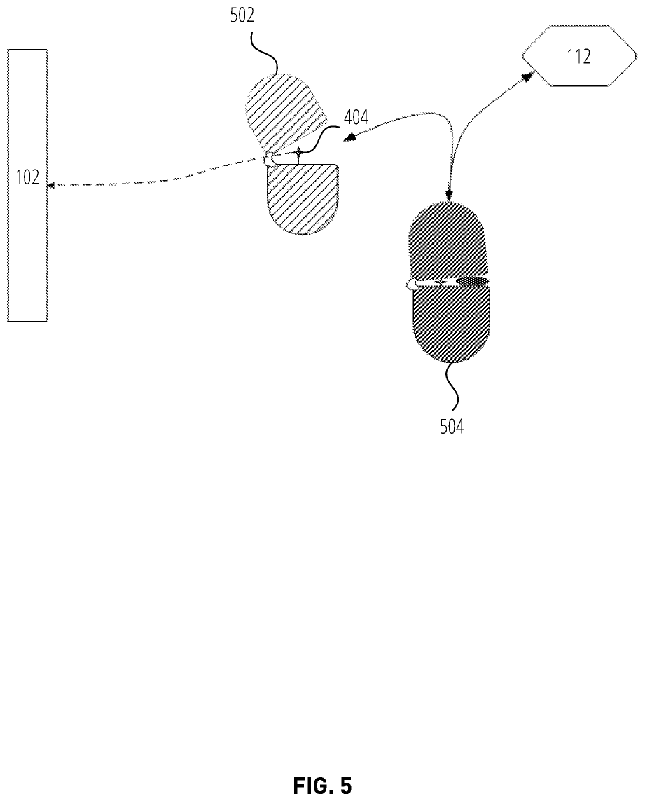

For a GGBP or set of GGBPs that suitably covers the physiological glucose concentration range, an electroactive redox molecule may be introduced to a specific location on the GGBP protein(s) as disclosed herein. Such electroactive redox molecules are referred to herein as redox mediators. The one or more redox mediator may create a measurable electric signal, when exposed. The redox mediator may be introduced to a location on the GGBP having accessibility dependent on the GGBP's conformational state. In certain implementations, the redox mediator may be more accessible (i.e., active or relatively more active) when GGBP is in the glucose-bound state. In other implementations, the redox mediator may be more accessible (i.e., active or relatively more active) when GGBP is in the unbound state. FIGS. 4A and 4B illustrate one such example implementation. FIG. 4A illustrates an engineered GGBP 402 in the unbound conformation having a redox mediator 404. FIG. 4B illustrates an engineered GGBP 402 in the glucose-bound conformation having a redox mediator 404. In this example, the redox mediator 404 is attached to a portion of the engineered GGBP 402, specifically a portion of the C-terminal domain 206, that becomes inaccessible when glucose 210 binds, as illustrated in FIG. 4B. Thus, the glucose-bound conformation versus unbound conformation of the engineered GGBP may be reflected in a change in electrical signal due to the accessibility, and therefore activity, of the redox mediator.

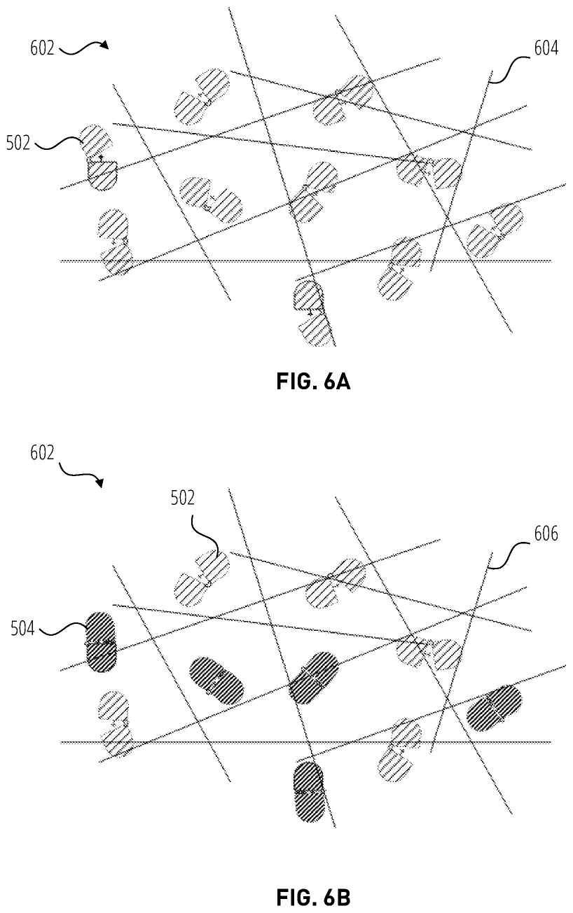

FIG. 5 illustrates an example scheme of conformation-dependent signal propagation to an electrode 102. In the illustrated example, the redox mediator 404 is accessible, and therefore active (or relatively more active), when the GGBP is in its unbound conformation 502. When active (or relatively more active), redox mediator 404 generates a signal that may be detected by electrode 102, as indicated by the dashed arrow. However, unbound GGBP 502 may reversibly bind with glucose 112 to form bound GGBP 504. The redox mediator 404 is not accessible, and thus not active (or only partially active) when GGBP is in its bound conformation 504. Consequently, when GGBP is in its bound conformation 504, the redox mediator 404 may not create a signal detectable by electrode 102. As discussed herein, the electrode 102 may include a nanowire network.

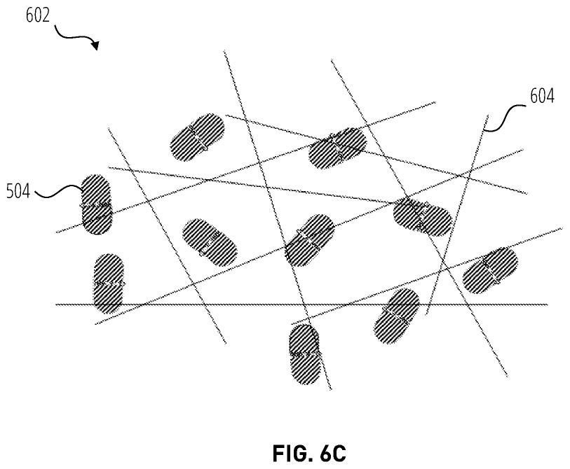

Engineered GGBPs may be embedded in a conductive hydrogel and/or polymer matrix. The conductive hydrogel or polymer matrix may also incorporate a nanowire network. Synthesis of such nanowire networks within hydrogels or polymer matrices is discussed in detail by Shi, Q. et al., Kinetically controlled synthesis of AuPt bi-metallic aerogels and their enhanced electrocatalytic performances, J. Mater. Chem. A 5, 19626-31 (2017), and Shi, Q. et al., Mesoporous Pt Nanotubes as a Novel Sensing Platform for Sensitive Detection of Intracellular Hydrogen Peroxide, Acs. Appl. Mater. Inter. 7, 24288-95 (2015), incorporated herein by reference. FIGS. 6A-6C illustrate an example of GGBPs embedded in a conductive hydrogel 602. There is a nanowire network 604 embedded in the conductive hydrogel 602. Also embedded are several engineered GGBPs according to the example illustrated in FIGS. 4A and 4B, where the redox mediator 404 is exposed while engineered GGBP 402 is unbound. FIG. 6A diagrams a situation in which the population of GGBPs are all unbound GGBPs 502. Thus, all redox mediators 404 are exposed and signal strength is at its fullest.

In FIG. 6B, half the GGBPs are unbound GGBP 502 and half are bound GGBP 504. Thus, signal strength due to redox mediator 404 may be less than that of FIG. 6B. In FIG. 6C, all GGPBs are unbound GGBPs 502. In this example, signal strength is at its lowest.

Nanowire networks may enhance sampling of the conformation-dependent electrical signal generated by the redox mediator on the GGBP protein. Nanowire networks may also increase the signal density due to the redox molecule, which in turn may allow for a smaller sensor size.

Such nanowire networks, in conjunction with engineered GGBP, may measure within the range of potential of approximately −1 to +1 V vs Ag/AgCl, approximately −0.75 to +0.75 V vs Ag/AgCl, approximately −0.5 to +0.5 V vs Ag/AgCl, approximately −0.4 to +0.4 V vs Ag/AgCl, approximately −0.3 to +0.3 V vs Ag/AgCl, approximately −0.25 to +0.25 V vs Ag/AgCl, approximately −0.2 to +0.2 V vs Ag/AgCl, or any value or range within or bounded by any of these ranges or values, although values outside these values or ranges can be used in some cases.

### Addition of Electroactive Molecules to a Binding Protein

In some aspects, a single site on the binding protein is engineered to incorporate a redox mediator. For example, a site of attachment may be chosen such that, when the GGBP undergoes a glucose-dependent conformation change, the redox mediator will either be occluded or accessible. Thus, the redox molecule may generate a glucose-dependent redox signal depending on GGBP glucose binding that can be measured, for example by cyclic voltammetry. Suitable redox molecules may include, but are not limited to, Ru(II) cofactor, porphyrin, and quinones. Redox molecules may be attached covalently to a binding protein, for example GGBP. Engineering of hinged binding proteins is discussed further by Benson, D. E. et al., Design of Bioelectronic Interfaces by Exploiting Hinge-Bending Motions in Proteins, Science 293, 1641-44 (2001), incorporated herein by reference.

With a higher concentration of glucose, more engineered GGBP molecules within a sensor in accordance with the present disclosure will be in the bound state. In aspects where the redox molecule is accessible when the engineered GGBP binds glucose, there may be a corresponding increase in electrical signal measured by the nanowire network as glucose concentration increases. In other aspects, where the redox molecule is inaccessible when the engineered GGBP is bound to glucose, there may be a corresponding decrease in electrical signal measured by the nanowire network as glucose concentration increases.

In certain aspects, two electroactive molecules may be attached to an engineered GGBP. One of these molecules may be a redox mediator as previously discussed. The second molecule, referred to herein as an affecter molecule, may shift the activity of the redox mediator when in proximity to the redox mediator. Each of the redox mediator and the affecter molecule may be attached to different amino acid sites on the GGBP. The amino acid sites may be chosen such that the distance between the amino acid sites changes due to the GGBP's conformational change when glucose binds. For example, the two sites may be relatively close together in the bound conformation but relatively far apart in the unbound conformation. In such aspects, the affecter molecule may be proximate to the redox mediator when the GGBP binds glucose and not proximate when the GGBP is not bound to glucose. In another example, the locations may be chosen such that the two sites are relatively far apart in the bound conformation but relatively close in the unbound conformation. In such aspects, the affecter molecule may be proximate to the redox mediator when the GGBP is not bound to glucose and not proximate the redox mediator when the GGBP is bound to glucose. These aspects may shift the redox mediator's peak potential that can be measured, for example by cyclic voltammetry.

### Aspects of an Analyte-Binding Sensor

A sensor in accordance with the present disclosure may address the shortcomings of enzymatic sensors. For example, because such a sensor depends on binding to glucose rather than conversion of glucose, local physiological glucose concentration may remain substantially unchanged. In the same vein, such a sensor may not create or may create fewer reactive byproducts, because GGBPs merely bind glucose rather than converting glucose to a different molecule.

Relative to enzymatic glucose sensors, a sensor in accordance with present disclosure may have a higher signal-to-noise ratio. For example, the sensor of the present disclosure may not require a glucose limiting layer to limit the concentration of glucose relative to another reactant, for example oxygen, of the glucose conversion reaction. The molecules involved in GGBP binding of glucose are (1) GGBP and (2) glucose. Thus, there may be no need to limit glucose concentration within the sensor.

The redox molecule can be chosen such that it is outside the electrochemical oxidation range of all, substantially all, a majority of, or many physiological electrochemical interferents. This includes exogenous electrochemical interferents, such as acetaminophen. A sensor in accordance with the present disclosure may therefore have minimal and/or less signal disruption due to electrochemical interferents. The sensor's signal may be entirely, substantially, or at least in part due to binding of glucose to the engineered GGBP.

Relatedly, with a higher signal-to-noise ratio, and depending on the glucose-correlated signal amplitude, a sensor in accordance with the present disclosure may be smaller than enzymatic sensors. For example, such a sensor may be approximately 100 μm long, which is smaller than glucose sensors currently on the market.

In certain aspects, the configuration of the sensor is relatively simple: it includes a hydrogel having a nanowire network and engineered GGBPs. Enzymatic glucose sensors may have several layers, for example a sensing electrode, an enzyme layer, a glucose limiting layer, and a blocking layer. Manufacturing a sensor in accordance with the present disclosure may be simpler relative to manufacturing a sensor having several layers. A simple manufacturing process may, in turn, result in a more reproducible and scalable production process. Because the number of components required for constructing such a sensor is small, the complexity of algorithms required to convert current measurements to concentration estimates may also be reduced substantially. In effect, the simplicity of the sensor design may reduce the number of parameters such an algorithm may need to address.

Sensor calibration may be relatively simple for a sensor in accordance with the present disclosure. For example, absolute changes due to glucose and sensor zero response can be established and/or calibrated before sensor fabrication. This pre-fabrication calibration can be translated to an implanted sensor. It may not be necessary to determine or guess unknown parameters after implantation, for example the effect of certain electrochemical interferents, as is the case for enzyme-based electrochemical CGM sensors.

A sensor in accordance with the present disclosure may have a longer sensing life than an enzymatic sensor. Because a sensor in accordance with the present disclosure does require enzymatic action, there are no, or substantially no, reactive oxygen species (ROS) generated by sensing. Most of the measurements by such a sensor may be performed at potentials near 0 V, for example from −0.2 to +0.2 V vs Ag/AgCl. Potentials near 0 V may oxidize fewer potential electrochemical interferents, resulting in fewer ROS. Because a sensor in accordance with the present disclosure produces fewer ROS due to a lack of enzymatic action and a measuring potential close to 0V, the life of the sensor may be relatively long. For example, such a sensor may have a sensing life of up to or greater than 30 days.

What is claimed is:

1. A sensor comprising:
a first binding protein configured to have a first conformation and a second conformation, wherein the first binding protein is in the first conformation when there is an analyte bound to a binding site of the first binding protein, and the first binding protein is in the second conformation when there is no analyte bound to the binding site of the first binding protein,
wherein an electric signal is generated when the first binding protein is in the first conformation, the first binding protein comprising a binding site; and
a sensing electrode.

2. The sensor of claim 1, comprising a redox mediator configured to create the electrical signal,
wherein
the redox mediator is not active or partially active when the first binding protein is in the first conformation,
the redox mediator is active or relatively more active in comparison to the first conformation when the first binding protein is in the second conformation.

3. The sensor of claim 2, further comprising:
an affecter molecule configured to shift the electrical activity of the redox mediator when the redox mediator and the affecter molecule are in proximity;
wherein the redox mediator and affecter molecule are not in proximity when the first binding protein is in the first conformation,
the redox mediator and affecter molecule are in proximity when the first binding protein is in the second conformation.

4. The sensor of claim 2, wherein the redox mediator is a Ru(II) cofactor, a porphyrin, or a quinone.

5. The sensor of claim 1, the first binding protein comprising a glucose binding protein.

6. The sensor of claim 5, wherein the glucose binding protein has a binding constant between about 100 nM to about 200 mM.

7. The sensor of claim 5, wherein the glucose binding protein has a binding constant between about 2 mM to about 22 mM.

8. The sensor of claim 1, wherein the analyte comprises glucose.

9. The sensor of claim 8, wherein the binding site of the first binding protein comprises a glucose binding site.

10. The sensor of claim 1, further comprising a second binding protein, wherein the first binding protein and second binding protein do not have the same binding constant.

11. The sensor of claim 1, wherein the sensing electrode comprises a nanowire network.

12. The sensing electrode of claim 1, the sensing electrode comprising a hydrogel.

13. A method of measuring a concentration of an analyte, comprising:
exposing the sensor of claim 1 to a sample fluid; and
measuring an electrical signal generated from the sensor.

14. The method of claim 13, wherein measuring the electrical signal comprises using an electrode comprising a nanowire network.

15. The method of claim 13, wherein the analyte comprises glucose.

16. The method of claim 13, wherein the binding protein comprises a glucose binding protein.

17. The method of claim 13, wherein the binding site comprises a glucose binding site.

18. The method of claim 13, wherein the electrical signal is generated by a redox mediator.

19. The method of claim 18, wherein
the redox mediator is not active or partially active when the binding protein is in the first conformation,
the redox mediator is active or relatively more active in comparison to the first conformation when the binding protein is in the second conformation.

20. The method of claim 19, further comprising an affecter molecule configured to shift the electrical activity of the redox mediator when the redox mediator and the affecter molecule are in proximity,
wherein the redox mediator and affecter molecule are not in proximity when the binding protein is in the first conformation, and
the redox mediator and affecter molecule are in proximity when the binding protein is in the second conformation.
